(19) United States
(12) Patent Application Publication
(10) Pub. No.: US 2025/0235615 A1
(43) Pub. Date: Jul. 24, 2025

(54) Multi-Chamber Syringe for Sequential Delivery of Fluids and Methods of Use

(71) Applicant: Becton, Dickinson and Company, Franklin Lakes, NJ (US)

(72) Inventors: Rahul Malviya, Salmon Arm (CA); Praveen Nalawade, Bangaluru (IN); Srimannarayana Myla, Bengaluru (IN)

(21) Appl. No.: 18/853,901

(22) PCT Filed: Mar. 31, 2023
(86) PCT No.: PCT/US23/17060

§ 371 Date: Oct. 3, 2024

Related U.S. Application Data
(60) Provisional application No. 63/327,185, filed on Apr. 4, 2022.

(51) Int. Cl. A61M 5/19; A61M 5/178; A61M 5/315

(52) U.S. Cl. CPC A61M 5/19; A61M 5/31511; A61M 2005/1787; A61M 2202/0478

(57) ABSTRACT

A multi-chamber syringe for sequential expulsion of a first fluid followed by a second fluid includes a barrel, a first stopper, and a second stopper. The barrel includes a proximal end, a distal end having a fluid port, and a sidewall. The first stopper including a channel is slidably positioned in the barrel, such that a first chamber configured to contain the first fluid is provided between the distal end of the barrel and the first stopper. The second stopper is slidably positioned in the barrel proximal to the first stopper, such that a second chamber configured to contain the second fluid is provided between the first stopper and the second stopper. Rotation of the first stopper relative to the syringe barrel establishes fluid communication between the second chamber and the first chamber through the channel of the first stopper.

### CROSS-REFERENCE TO RELATED APPLICATION

The present application claims priority to U.S. Provisional Patent Application Ser. No. 63/327,185, entitled “Multi-Chamber Syringe for Sequential Delivery of Fluids and Methods of Use”, filed Apr. 4, 2022, the entire disclosure of which is hereby incorporated by reference in its' entirety.

### BACKGROUND OF THE INVENTION

### Field of the Invention

The present disclosure relates generally to syringes for expelling multiple fluids and, in particular, to a syringe for sequential expulsion of a first fluid, such as a medical fluid, followed by a second fluid, such as a flush solution.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Many medical facilities implement sterile practices and protocols to ensure that VADs are used properly and do not become sealed or infected. These protocols often include sterilizing the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition.

These flush procedures are intended to confirm catheter patency, avoid drug incompatibilities, ensure that the complete drug dose is administered to the patient, prevent thrombus formation, and minimize a risk of bloodstream infections caused by contamination of the VAD. Further, flushing can prevent build-up of deposits of blood, blood residue, and IV drugs within a catheter or other VAD device. Such build-up can cause partial or complete blockage of the fluid pathway in a catheter system, which may require a practitioner to perform certain expensive and potentially dangerous processes for purging the affected catheter or may require the practitioner to exchange the blocked catheter with a new catheter. Often, such blockages lead to interruptions in therapy, which may compromise patient care. The build-up of residue within a catheter may also increase infection risk.

During administration of a drug or another medical fluid, a catheter or VAD is usually flushed both before and after delivery of the fluid from a syringe or from another drug delivery device to the VAD. In some cases, catheters can be flushed using syringe assemblies that are pre-filled with various fluids (referred to herein as flush solutions). The size of the pre-filled syringe used to flush the catheter varies based, for example, on a size and length of the catheter. Typically, pre-filled syringes for flushing a catheter or IV line contain a volume of about 1 mL, 3 mL, 5 mL, or 10 mL of flush solution. Many different syringes commercially available from different manufacturers can be used for providing the flush solution through a catheter or IV line of a patient including, for example, the BD PosiFlush™ pre-filled saline syringe by Becton, Dickinson and Company.

In order to perform a flushing procedure, a practitioner connects the pre-filled flushing syringe to the VAD and advances a plunger rod into a barrel of the pre-filled syringe, which expels the flush solution through a nozzle or fluid port of the pre-filled syringe to the VAD. After flushing, the pre-filled syringe is disconnected from the VAD so that the practitioner can administer a dose of the medical fluid. The medical fluid is conventionally contained in a vial and is drawn into a separate syringe which, after being filled with the medical fluid, is connected to the VAD. The medical fluid is then injected to the VAD by advancing a plunger rod through the syringe containing the medical fluid. After the medical fluid is injected to the VAD, the syringe is disconnected from the VAD and a second pre-filled flushing syringe is used to flush the VAD. Flushing the VAD after administering the medical fluid confirms patency of the catheter and advances any remaining medical fluid in the catheter to the patient ensuring that a full dose of the medical fluid is delivered to the patient.

Connecting or disconnecting a syringe or another device from the VAD introduces portions of the VAD to an unsterile outside environment, which increases a possibility that the VAD will become contaminated and/or will be a source of a bloodstream infection. In order to decrease a risk of bloodstream infection and to ensure VADs are used and maintained correctly, standards of practice require disinfecting and cleaning any fluid ports or connectors of the VAD each time that a new syringe is connected to the VAD. Accordingly, reducing a number of syringes that must be connected to the VAD during a drug delivery procedure simplifies drug administration by reducing a number of times that portions of the VAD must be disinfected and cleaned. Reducing the number of syringes or other fluid delivery devices used during a procedure also reduces opportunities for portions of the VAD to become contaminated, thereby reducing the risk for bloodstream infections. The syringes and methods of the present disclosure provide simplified drug administration procedures by reducing the number of syringes used during such procedures, thereby reducing a risk of contamination and bloodstream infection.

### SUMMARY OF THE INVENTION

According to an aspect of the present disclosure, a multi-chamber syringe for sequential expulsion of at least a first fluid followed by a second fluid includes a barrel, a first stopper, and a second stopper. The barrel includes a proximal end, a distal end having a fluid port for expulsion of the first fluid and the second fluid from the barrel, and a sidewall extending between the proximal end and the distal end of the barrel. The first stopper including at least one channel is slidably positioned in the barrel, such that a first chamber configured to contain the first fluid is provided between the distal end of the barrel and the first stopper. The second stopper is slidably positioned in the barrel proximal to the first stopper, such that a second chamber configured to contain the second fluid is provided between the first stopper and the second stopper. Rotation of the first stopper relative to the syringe barrel establishes fluid communication between the second chamber and the first chamber through the at least one channel of the first stopper.

According to another aspect of the disclosure, a pre-filled flushing syringe includes any of the previously described multi-chamber syringes and a predetermined volume of the second fluid disposed in the second chamber of the pre-filled syringe. The pre-filled syringe is provided with the first chamber fluidly isolated from the second chamber, thereby containing the predetermined volume of the second fluid within the second chamber of the pre-filled syringe.

According to another aspect of the disclosure, a method for sequential expulsion of fluids from a multi-chamber syringe includes a step of moving the first stopper and the second stopper of any of the previously described syringes through the barrel in a distal direction, thereby expelling the first fluid from the barrel through the fluid port of the barrel. The method further includes rotating the first stopper, thereby establishing fluid communication between the second chamber and the first chamber; and moving the second stopper through the barrel in the distal direction towards the first stopper, thereby causing the second fluid to pass from the second chamber, through the at least one channel of the first stopper, the first chamber, and the fluid port of the barrel.

Non-limiting illustrative examples of embodiments of the present disclosure will now be described in the following numbered clauses.

Clause 1: A multi-chamber syringe for sequential expulsion of at least a first fluid followed by a second fluid, the syringe comprising: a barrel comprising a proximal end, a distal end comprising a fluid port for expulsion of the first fluid and the second fluid from the barrel, and a sidewall extending between the proximal end and the distal end of the barrel; a first stopper comprising at least one channel, the first stopper being slidably positioned in the barrel with a first chamber configured to contain the first fluid between the distal end of the barrel and the first stopper; and a second stopper slidably positioned in the barrel proximal to the first stopper with a second chamber configured to contain the second fluid between the first stopper and the second stopper, wherein rotation of the first stopper relative to the syringe barrel establishes fluid communication between the second chamber and the first chamber through the at least one channel of the first stopper.

Clause 2: The syringe of clause 1, wherein movement of the first stopper and the second stopper through the barrel in a proximal direction aspirates fluid into the first chamber through the fluid port of the barrel.

Clause 3: The syringe of clause 1 or clause 2, wherein, in a filled position with the first fluid in the first chamber and the second fluid in the second chamber, the first stopper is spaced apart from the second stopper and the second chamber is not in fluid communication with the first chamber.

Clause 4: The syringe of clause 3, wherein advancing the first stopper and the second stopper through the barrel in a distal direction from the filled position to an intermediate position expels the first fluid from the first chamber through the fluid port of the barrel.

Clause 5: The syringe of clause 4, wherein, in the intermediate position, the first stopper is in a distal-most position within the syringe barrel and all or substantially all of the first fluid is expelled from the first chamber.

Clause 6: The syringe of clause 4 or clause 5, wherein, with the stoppers in the intermediate position, rotating the first stopper establishes fluid communication between the second chamber and the first chamber through the at least one fluid channel of the first stopper.

Clause 7: The syringe of clause 6, wherein, with the second chamber in fluid communication with the first chamber, movement of the second stopper axially through the barrel towards the first stopper moves the second fluid from the second chamber, through the at least one channel of the first stopper, the first chamber, and the fluid port of the barrel, thereby expelling the second fluid from the barrel.

Clause 8: The syringe of any of clauses 1-7, wherein the barrel comprises at least one of polyester, polycarbonate, polypropylene, polyethylene, polyethylene terephthalate, or acrylonitrile butadiene styrene.

Clause 9: The syringe of any of clauses 1-8, wherein the first stopper and/or the second stopper comprise a thermoplastic elastomer.

Clause 10: The syringe of clause 9, wherein the first stopper and/or the second stopper comprise a proximal end, a distal end, an outer surface extending between the proximal end and the distal end, and at least one annular rib protruding from the outer surface that seals against an inner surface of the sidewall of the barrel.

Clause 11: The syringe of any of clauses 1-10, wherein the barrel comprises at least one widened portion, wherein a radial distance between the inner surface of the sidewall of the barrel in the at least one widened portion and a central axis of the barrel is greater than the radial distance between the inner surface of the sidewall and the central axis of the barrel at other portions of the barrel.

Clause 12: The syringe of clause 11, wherein the at least one widened portion has an arcuate width of from 45 degrees to 70 degrees about the central axis of the barrel.

Clause 13: The syringe of clause 12, wherein the at least one widened portion of the barrel comprises a first widened portion on one side of the barrel and a second widened portion on an opposite side of the barrel, such that the barrel is vertically symmetrical.

Clause 14: The syringe of any of clauses 11-13, wherein the at least one channel through the first stopper comprises an inlet and an outlet, and wherein fluid flow through the at least one channel occurs only when the outlet of the at least one channel is in the at least one widened portion of the barrel.

Clause 15: The syringe of any of clauses 11-14, wherein the second chamber and the first chamber are not in fluid communication through the at least one channel of the stopper when the outlet of the at least one channel is not in the at least one widened portion of the barrel.

Clause 16: The syringe of any of clauses 1-15, wherein the at least one channel of the first stopper comprises a first cutout section on one side of the stopper and a second cutout section on an opposite side of the first stopper from the first cutout section, such that the first stopper is vertically symmetrical.

Clause 17: The syringe of any of clauses 1-15, wherein the at least one channel comprises at least one enclosed passage extending from an inlet on a proximal end of the first stopper to an outlet on a side surface of the first stopper.

Clause 18: The syringe of clause 17, wherein the at least one enclosed passage comprises a first enclosed passage on one side of the first stopper and a second enclosed passage radially on an opposite side of the first stopper from the first enclosed passage, such that the first stopper is vertically symmetrical.

Clause 19: The syringe of any of clauses 1-18, wherein the first stopper comprises a recessed groove on the distal end of the first stopper positioned to permit fluid flow past the first stopper to the fluid port of the barrel.

Clause 20: The syringe of any of clauses 1-19, further comprising at least one guide member fixed to the first stopper for rotating the first stopper relative to the barrel.

Clause 21: The syringe of clause 20, wherein a distal end portion of the guide member is received within a cavity of the first stopper, and wherein the guide member comprises at least one axially extending ridge that engages an axial slot of the cavity, thereby fixing the guide member to the first stopper.

Clause 22: The syringe of clause 20 or clause 21, wherein the second stopper comprises a through-hole, and wherein the at least one guide member extends through the through-hole, allowing the second stopper to slide along the guide member when the second stopper is moved through the barrel in a distal direction.

Clause 23: The syringe of any of clauses 20-22, further comprising a plunger rod connected to the second stopper for moving the second stopper and/or the first stopper through the barrel.

Clause 24: The syringe of clause 23, further comprising a removable plunger cap engaged between the plunger rod and the barrel for preventing movement of the plunger rod until the removable plunger cap is removed from the syringe.

Clause 25: The syringe of clause 23 or clause 24, wherein a distal end of the plunger rod is engaged in an annular cavity on the proximal end of the second stopper.

Clause 26: The syringe of any of clauses 23-25, wherein the plunger rod comprises an elongated recess that receives the guide member as the second stopper moves through the barrel in a distal direction towards the first stopper.

Clause 27: The syringe of clause 26, wherein the plunger rod comprises an inwardly protruding tab that engages the at least one guide member so that rotation of the plunger rod rotates the at least one guide member and the first stopper.

Clause 28: The syringe of any of clauses 23-27, wherein the barrel comprises at least one rotation restrictor that engages the plunger rod preventing rotation of the plunger rod, stoppers, and at least one guide member unless the first stopper is at a distal-most position in the barrel.

Clause 29: A pre-filled flushing syringe, comprising: the syringe of any of clauses 1-28; and a predetermined volume of the second fluid disposed in the second chamber of the pre-filled syringe, wherein the pre-filled syringe is provided with the first chamber fluidly isolated from the second chamber, thereby containing the predetermined volume of the second fluid within the second chamber of the pre-filled syringe.

Clause 30: The pre-filled syringe of clause 29, wherein the second fluid comprises a saline and/or heparin flush solution.

Clause 31: The pre-filled syringe of clause 29 or clause 30, wherein the syringe further comprises a plunger rod connected to the second stopper for moving the second stopper and/or the first stopper through the barrel.

Clause 32: The pre-filled syringe of clause 31, further comprising a removable plunger cap engaged between the plunger rod and the barrel of the syringe for preventing movement of the plunger rod until the cap is removed, thereby maintaining fluid isolation of the first chamber and the second chamber until the plunger cap is removed.

Clause 33: A method for sequential expulsion of fluids from the syringe of any of clauses 1-28, the method comprising: moving the first stopper and the second stopper of the syringe of any of clauses 1-28 through the barrel in a distal direction, thereby expelling the first fluid from the barrel through the fluid port of the barrel; rotating the first stopper thereby establishing fluid communication between the second chamber and the first chamber; and moving the second stopper through the barrel in the distal direction towards the first stopper thereby causing the second fluid to pass from the second chamber, through the at least one channel of the first stopper, the first chamber, and the fluid port of the barrel.

Clause 34: The method of clause 33, wherein the syringe is provided partially prefilled containing a predetermined volume of the second fluid in the second chamber.

Clause 35: The method of clause 34, further comprising drawing the first fluid into the first chamber of the barrel of the partially prefilled syringe by moving the stoppers through the barrel in a proximal direction.

Clause 36: The method of clause 35, wherein drawing the first fluid into the first chamber comprises attaching a needle to the fluid port of the syringe barrel, inserting the needle into a fluid container, and drawing the first fluid from the fluid container to the first chamber of the syringe.

Clause 37: The method of any of clauses 33-36, further comprising attaching at least one patient line to the fluid port of the syringe prior to moving the stoppers through the barrel in the distal direction, wherein the fluid expelled from the syringe passes to the patient through at least one patient line.

Clause 38: The method of any of clauses 33-37, wherein the first fluid comprises a therapeutic agent and the second fluid comprises a flush solution, such as saline solution and/or heparin lock flush solution.

### BRIEF DESCRIPTION OF THE DRAWINGS

FIG. 1A is a perspective view of a multi-chamber syringe, according to an aspect of the present disclosure.

FIG. 1B is a perspective exploded view of the multi-chamber syringe of FIG. 1A.

FIG. 2A is a side view of the multi-chamber syringe in an initial or partially filled position of FIG. 1A.

FIG. 2B is a cross-sectional view of the multi-chamber syringe of FIG. 1A in a filled position.

FIG. 2C is a cross-sectional view of the multi-chamber syringe of FIG. 1A in an intermediate position with a filled second chamber and an empty first chamber.

FIG. 2D is a cross-sectional view of the multi-chamber syringe of FIG. 1A in the intermediate position after rotating the first stopper to establish fluid communication between the second chamber and the first chamber.

FIG. 2E is a cross-sectional view of the multi-chamber syringe of FIG. 1A in a final position after expulsion of the first fluid and the second fluid from the syringe.

FIG. 3A is a proximal perspective view of the first stopper of the syringe of FIG. 1A, according to an aspect of the present disclosure.

FIG. 3B is a distal perspective view of the first stopper of FIG. 3A.

FIG. 3C is a cross-sectional view of the first stopper of FIG. 3A.

FIG. 4A is a perspective view of the second stopper of the syringe of FIG. 1A, according to an aspect of the present disclosure.

FIG. 4B is a cross-sectional view of the second stopper of FIG. 4A.

FIG. 5A is a perspective view of a guide member of the syringe of FIG. 1A, according to an aspect of the present disclosure.

FIG. 5B is a cross-sectional view of the guide member of FIG. 5A.

FIG. 6A is a perspective view of the barrel of the syringe of FIG. 1A, according to an aspect of the present disclosure.

FIG. 6B is a cross-sectional view of a distal portion of the barrel of FIG. 6A.

FIG. 6C is a perspective view of the proximal end of the barrel of FIG. 6A.

FIG. 7A is a perspective view of the plunger rod of the syringe of FIG. 1A, according to an aspect of the present disclosure.

FIG. 7B is a perspective view of the distal end of the plunger rod of FIG. 7A.

FIG. 8 is a flow chart illustrating a method for sequential expulsion of fluids from a multi-chamber syringe, according to an aspect of the present disclosure.

FIG. 9A is a cross-sectional view of another example of a multi-chamber syringe in an intermediate position with a filled second chamber and an empty first chamber.

FIG. 9B is a cross-sectional view of the multi-chamber syringe of FIG. 9A in the intermediate position after rotating the first stopper to establish fluid communication between the second chamber and the first chamber.

FIG. 9C is a cross-sectional view of the first stopper of the syringe of FIG. 9A.

FIG. 9D is another cross-sectional view of the first stopper of the syringe of FIG. 9A.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, that is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to multi-chamber syringes 10, 210 that expel a first fluid F1 followed by a second fluid F2 from a syringe barrel through a fluid port or nozzle of the syringe 10, 210 to, for example, a vascular access device. The first fluid F1 can be a medical fluid, which, as used herein, can refer to a medication, a total parenteral nutrient (TPN) liquid, or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect. The second fluid F2 can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringes 10, 210 of the present disclosure. The syringes 10, 210 can also be used for sequential delivery of a first medication or therapeutic agent followed by a different second medication or therapeutic agent.

The syringes 10, 210 of the present disclosure allow a practitioner, such as a medical technician, nurse, physician assistant, physician, or other trained or untrained clinicians or medical caregivers, to administer, for example, a medication followed by a flush solution without needing to change syringes between delivery of the first fluid and the second fluid. Beneficially, the sequential delivery of the medication followed by the flush solution occurs automatically through advancement of, for example, a plunger rod of the syringes 10, 210. As used herein, “advancement” of the plunger rod means that the practitioner is able to push the plunger rod in a distal direction, through a barrel of the syringe 10, 210, as a single stroke to expel the first fluid followed by the second fluid from the syringe barrel. The practitioner does not need to, for example, disconnect a syringe or another device from the VAD between delivery of the first fluid and the second fluid. Further, using the syringes 10, 210 of the present disclosure, the practitioner does not need to change a direction of movement of the plunger rod or press another component or mechanism of the syringe 10, 210 in order to perform the sequential delivery of the first fluid and the second fluid.

In some examples, the syringes 10, 210 are provided as a partially pre-filled syringe, where a chamber of the syringe 10, 210 is filled with a flush solution during manufacturing. The partially pre-filled syringe 10, 210 can include caps, clips, retainers, and/or other packaging to hold the plunger rod in place and to ensure that the flush solution does not leak from the partially pre-filled syringe 10, 210 at unexpected times, such as during transport.

The syringes 10, 210 of the present disclosure are also configured to allow the practitioner to aspirate a medical fluid into the syringe 10, 210 prior to fluid delivery to the patient. For example, the practitioner can insert a nozzle or needle of the syringe 10, 210 into a vial containing the medical fluid and then aspirate the medical fluid into a fluid chamber of the syringe 10, 210 by moving the plunger rod of the syringe 10, 210 in a proximal direction. After the medical fluid is aspirated into the fluid chamber of the syringe 10, 210, sequential delivery of the first fluid followed by the second fluid can occur by connecting the syringe 10, 210 to a fluid port of a VAD and then moving the plunger rod of the syringe 10, 210 in the distal direction, thereby expelling the first fluid followed by the second fluid from the syringe 10, 210 to the VAD.

By eliminating the need to clean or disinfect portions of the VAD between delivery of the first fluid and the second fluid, the syringes 10, 210 of the present disclosure simplify the fluid administration procedure, providing substantial time savings compared to conventional fluid administration practices. The syringes 10, 210 of the present disclosure also reduce infection risk and allow for flushing of the VAD immediately following administration of the medical fluid, which may prevent drug occlusion in the VAD. Further, the syringes 10, 210 of the present disclosure have zero dead space, meaning that all medical fluid contained in a first chamber of the syringe 10, 210 is flushed from the syringe 10, 210 as the second fluid (e.g., the flush solution) moves through the syringe 10, 210 to the VAD.

### Multi-Chamber Syringe for Sequential Drug Delivery

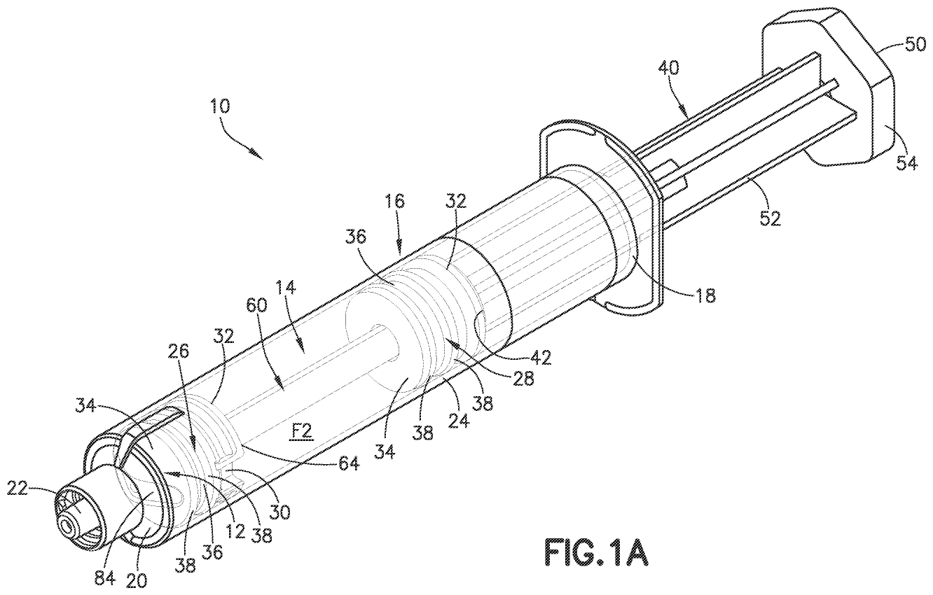

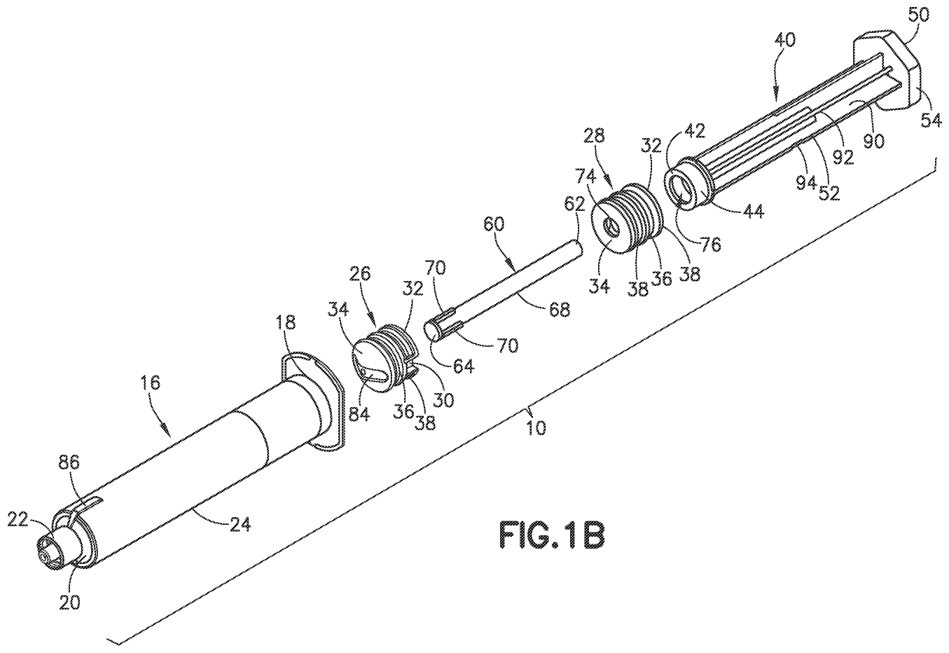

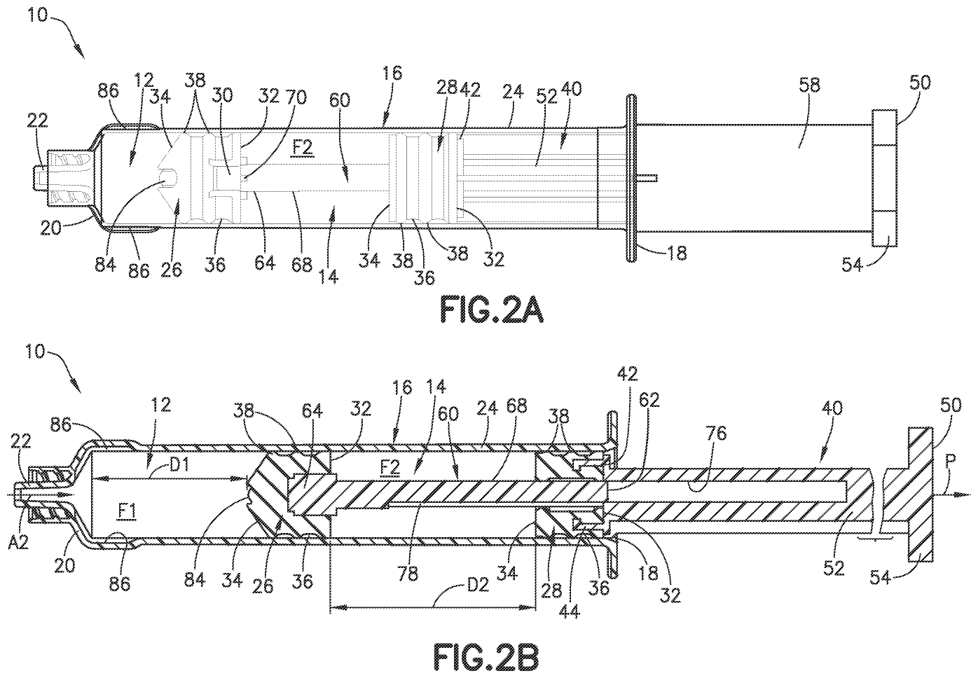

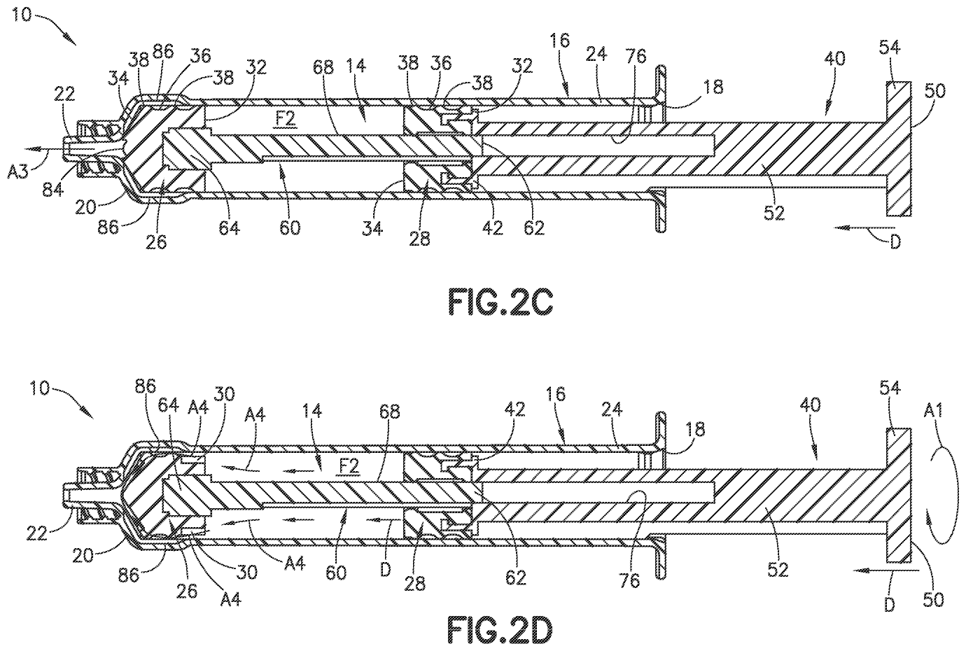

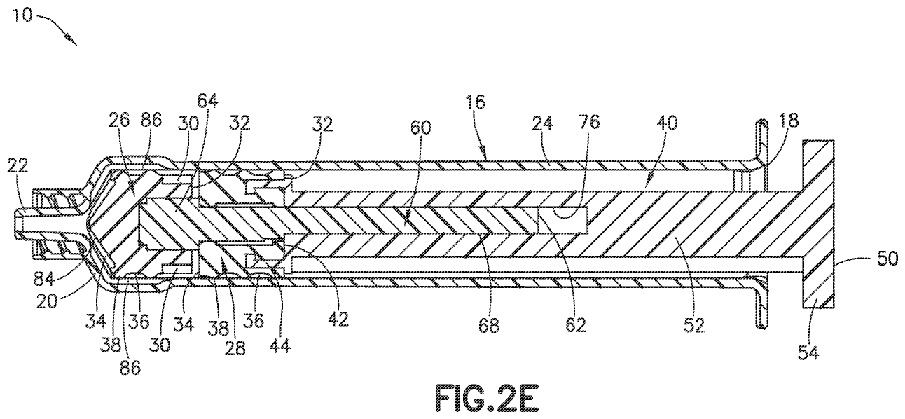

FIGS. 1A-2E illustrate an example of a multi-chamber syringe 10 for sequential expulsion of at least a first fluid F1 contained in the first chamber 12 followed by a second fluid F2 contained in a second chamber 14. As previously described, the first fluid F1 can be a medical fluid, such as a drug or another therapeutic agent intended for delivery to a patient through a VAD, such as a catheter or IV line. The second fluid F2 can be a flush solution, such as saline solution and/or an anticoagulant, such as heparin. The type of flush solution and amount of flush solution contained in the second chamber 14 may vary depending, for example, on the specific type of catheter or IV line being used. In some examples, the syringe 10 contains or is configured to contain between about 1 mL and 20 mL of the second fluid F2 or, preferably, between about 5 mL and about 10 ml of the second fluid F2.

In some examples, the syringe 10 comprises a barrel 16 having an open proximal end 18, a distal end 20 including a nozzle or fluid port 22 for expulsion of the first fluid F1 and the second fluid F2 from the barrel 16, and a sidewall 24 extending between the proximal end 18 and the distal end 20 of the barrel 16. The fluid port 22 of the barrel 16 can be a connector, such as a luer connector, threaded connector, or snap connector, configured to be connected to a needle for accessing, for example, an interior of a medical vial containing a medical fluid. The fluid port 22 of the syringe 10 can also be configured to be connected directly or indirectly to a fluid port, valve, or another terminal access portion of a VAD. For example, a common type of fluid port of a VAD is a pierceable septum or pre-slit septum made of rubber or another elastomeric material, which permits insertion of a sharp or blunt needle cannula in order to infuse fluids or to withdraw fluids from a catheter of the VAD. Another common fluid port of a VAD is a valve, which does not require a needle for accessing the VAD. Instead, the valve can be activated by a frusto-conically shaped tip of the syringe barrel 16 to provide fluid communication between the interior of the barrel 16 and the VAD.

In some examples, the barrel 16 of the syringe 10 can be substantially similar in shape, size, and configuration to barrels of syringes used for administering a flush solution to a VAD, as are known in the art. For example, the barrel 16 can be a cylindrical structure formed from a rigid thermoplastic material, such as polyester, polycarbonate, polypropylene, polyethylene, polyethylene terephthalate, acrylonitrile butadiene styrene or other injection moldable or formable resin materials, as are known in the art. Exemplary barrels for flush syringes are described, for example, in U.S. Patent Appl. Pub. No. 2020/0061297, entitled “Flush Syringe Assembly with Controlled Pulsatile Flushing,” which is incorporated herein by reference in its entirety.

The syringe 10 further comprises a distal or first stopper 26 and a proximal or second stopper 28 slidably positioned within the barrel 16 of the syringe 10. The stoppers 26, 28 separate the barrel 16 into the first chamber 12 and the second chamber 14. Specifically, as shown in FIG. 1A, the distal or first chamber 12 is between the distal end 20 of the barrel 16 and a distal end of the first stopper 26. The proximal or second chamber 14 is between a proximal end of the first stopper 26 and the second stopper 28.

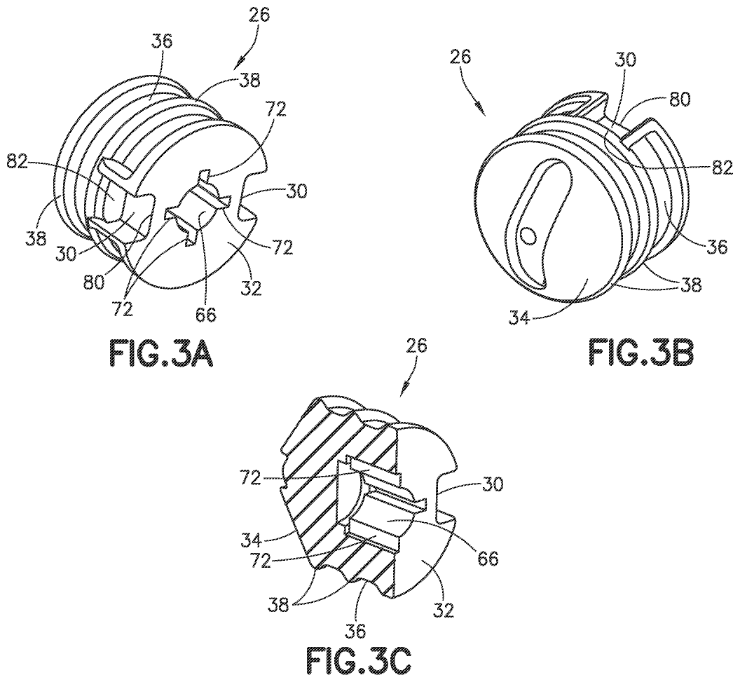

As shown, for example, in FIGS. 1A, 1B, 2D, and 2E, as well as in FIGS. 3A-3C, the first stopper 26 includes cutout sections or channels 30 that, when properly oriented, allow fluid to pass from the second chamber 14 to the first chamber 12. As described in further detail herein, rotation of the first stopper 26 relative to the syringe barrel 16 properly orients the channels 30 of the first stopper 26, thereby establishing the fluid communication between the second chamber 14 and the first chamber 12 through the channels 30 of the first stopper 26.

The stoppers 26, 28 include many features of conventional syringe stoppers or plungers, as are known in the art. For example, the stoppers 26, 28 can be formed from a thermoplastic elastomer material, such as polypropylene or polyethylene, as well as from synthetic or natural rubber (e.g., isoprene). The first stopper 26 and/or the second stopper 28 can include a proximal surface 32 or proximal end, a distal surface 34 or end, an outer peripheral surface 36 extending between the proximal surface 32 and the distal surface 34. In some examples, as with the first stopper 26 shown in FIGS. 1A-2E, the distal surface 34 or distal end of the stopper is can be conical or frusto-conical to assist in expelling fluid from the barrel 16 through the fluid port 22. Alternatively, the distal end or distal surface 34 of the stopper 26, 28 can be substantially flat, as with the second stopper 28. The stoppers 26, 28 can further include one or more annular ribs 38 protruding from the outer peripheral surface 36. The annular ribs 38 are configured to seal against an inner surface of the sidewall 24 of the syringe barrel 16, ensuring that fluid moves through the syringe barrel 16 in an expected manner. In some examples, the stoppers 26, 28 include multiple annular ribs 38 in order to improve stability and to prevent the stoppers 26, 28 from tilting, shifting, or otherwise deforming as they move through the syringe barrel 16.

The stoppers 26, 28 are configured to move through the syringe barrel 16 to aspirate fluid into the barrel 16 and to expel fluid from the barrel 16. For example, movement of the first stopper 26 and the second stopper 28 through the barrel 16 in a proximal direction, as shown by arrow P in FIG. 2B, aspirates the first fluid F1 into the first chamber 12 through the fluid port 22 of the barrel 16. In a similar manner, as described in further detail hereinafter, movement of the first stopper 26 and the second stopper 28 through the barrel 16 in a distal direction, as shown by arrow D in FIGS. 2C and 2D, expels the first fluid F1 from the first chamber 12 and the second fluid F2 from the second chamber 14 through the fluid port 22 of the barrel 16.

More specifically, the stoppers 26, 28 are configured to move between several positions during a fluid expulsion procedure. The syringe 10 may initially be provided in a partially filled position, as shown in FIG. 2A, with a second fluid F2, such as flush solution, in the second chamber 14. In this initial or partially filled position, the stoppers 26, 28 are spaced apart from one another by a distance D1 (shown in FIG. 2B) sufficient to contain a volume of the second fluid F2 of about 1 mL to 20 mL, or preferably about 5 mL to 10 mL. When ready for use, the practitioner can move the first stopper 26 to a distal-most position seated against the distal end 20 of the barrel 16. The practitioner can then move the stoppers 26, 28 in the proximal direction (shown by arrow P in FIG. 2B) to aspirate the first fluid F1 into the first chamber 12, thereby fully filling the syringe 10, as shown in FIG. 2B. As shown in FIG. 2B, when the syringe is fully filled, the first stopper 26 is a distance D2 from the distal end 20 of the barrel 16. Once the syringe 10 is fully filled, moving the stoppers 26, 28 in the distal direction (shown by arrow D in FIGS. 2C and 2D) moves the first stopper 26 towards the distal end 20 of the syringe 10, thereby expelling the first fluid F1 from the first chamber 12. The syringe 10 is shown in an intermediate position with the full second chamber 14 and the empty first chamber 12 in FIG. 2C. Next, fluid communication between the second chamber 14 and the first chamber 12 is established by rotating the first stopper 26 to properly align the channels 30 of the first stopper 26, as shown by the arrow A1 in FIG. 2D. The second fluid F2 is then expelled from the syringe 10 by moving the second stopper 28 towards the first stopper 26. Specifically, movement of the second stopper 28 towards the first stopper 26 causes the second fluid F2 to move from second chamber 14 to the first chamber 12, and then from the syringe barrel 16 through the fluid port 22. FIG. 2E shows the syringe 10 in an end-of-use position after the fluids F1, F2 are expelled from the syringe barrel 16. In this end-of-use position, the distal surface 34 of the second stopper 28 in contact with or nearly in contact with the proximal surface 32 of the first stopper 26.

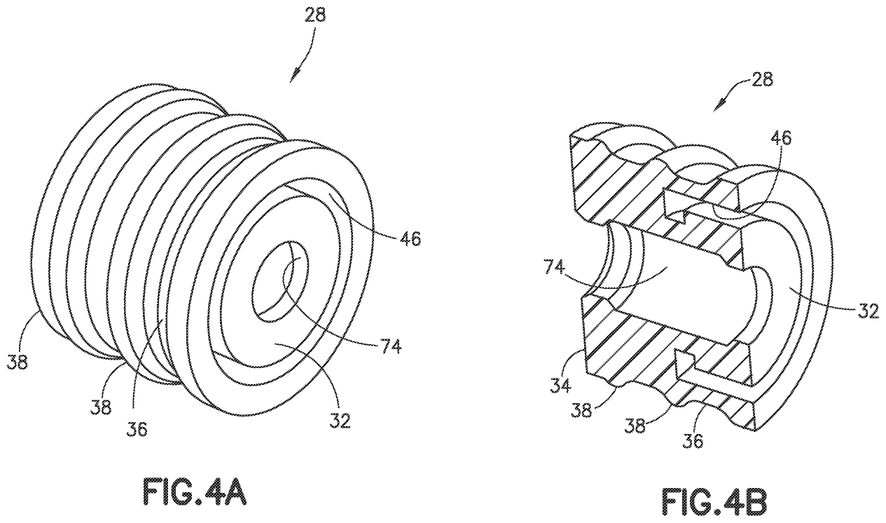

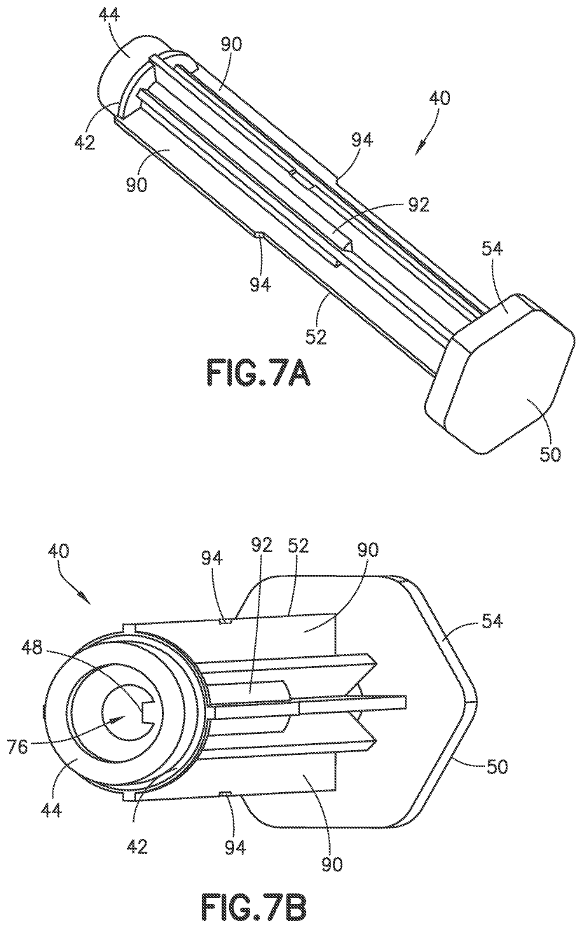

With continued reference to FIGS. 1A-2E, the syringe 10 further comprises a plunger rod 40 connected to the second stopper 28 for moving the second stopper 28 and the first stopper 26 through the syringe barrel 16. The plunger rod 40 can be, for example, an injection molded part formed from a rigid thermoplastic material, such as polyester, polycarbonate, polypropylene, polyethylene, polyethylene terephthalate, or another thermoplastic material, as are known in the art. The plunger rod 40 includes a distal end 42 engaged to the second stopper 28. For example, as most clearly seen in FIG. 7B, the distal end 42 of the plunger rod 40 can include an annular connector 44 that is inserted into a corresponding annular cavity 46 (shown in FIG. 4B) or slot on the proximal surface 32 of the second stopper 28. The plunger rod 40 can also include a tab 48 (shown in FIG. 7B) that extends radially inwardly from the annular connector 44 for maintaining positioning of the plunger rod 40 relative to other structures of the syringe 10. The plunger rod 40 also includes a proximal end 50 protruding proximally from the proximal end 18 of the syringe barrel 16 and a body 52 extending between the proximal end 50 and the distal end 42 of the plunger rod 40. The proximal end 50 of the plunger rod 40 can include a thumb press plate 54 for manipulating the plunger rod 40 to move the stoppers 26, 28 through the syringe barrel 16. The body 52 of the plunger rod 40 can have a variety of cross-sectional shapes and configurations within the scope of the present disclosure. For example, as shown in FIGS. 7A and 7B, the body 52 has a generally cross shaped cross-section. In other examples, the cross-section of the plunger rod 40 can be an I-beam shape, hollow circle, hollow square, hollow rectangle, or L-shaped. In some examples, as shown in FIG. 2A, the syringe 10 can also include a removable plunger cap 58 (shown in FIG. 2A) engaged between the thumb press plate 54 of the plunger rod 40 and the proximal end 18 of the barrel 16 for preventing movement of the plunger rod 40 until the removable plunger cap 58 is removed from the syringe 10.

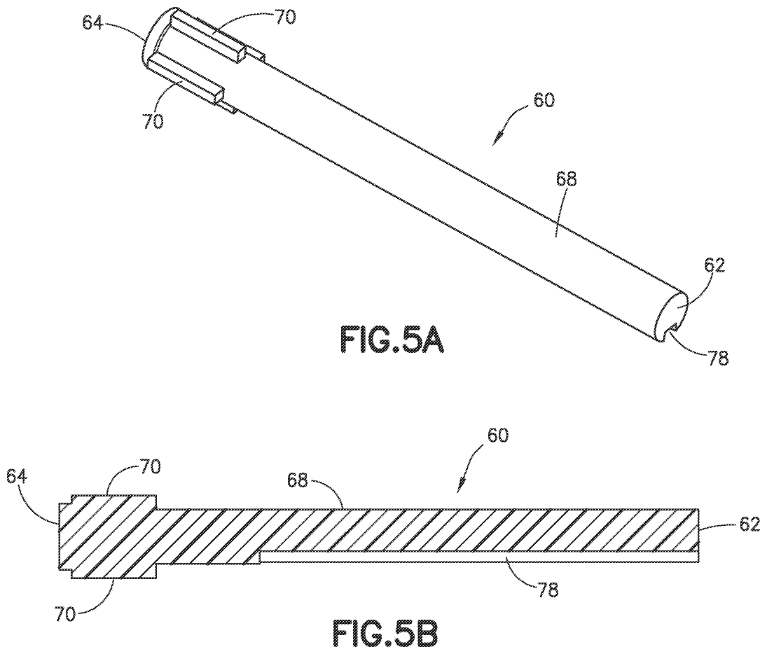

In some examples, the syringe 10 further comprises an elongated guide member 60 fixed to the first stopper 26 for rotating the first stopper 26 relative to the barrel 16. The guide member 60 can be formed from a rigid thermoplastic material, which may be the same material as the barrel 16 and/or the plunger rod 40, or a different material. As shown in FIGS. 5A and 5B, the guide member 60 includes a proximal end 62, a distal end 64 received within a cavity 66 (shown in FIGS. 3A-3C) of the first stopper 26, and a sidewall or side surface 68 extending between the proximal end 62 and the distal end 64 of the guide member 60. The guide member 60 can include radially extending locking protrusions or ridges 70 extending from the distal end 64 that engage corresponding slots 72 or recess in the cavity 66 of the first stopper 26, thereby fixing the guide member 60 to the first stopper 26. As shown in FIGS. 2A-2E, the guide member 60 passes through the second stopper 28. For example, the guide member 60 can pass through a through-hole 74 (shown most clearly in FIGS. 4A and 4B) in the second stopper 28, which allows the second stopper 28 to slide along the guide member 60 as the second stopper 28 is moved through the barrel 16 in a distal direction by the plunger rod 40.

In some examples, the plunger rod 40 includes an elongated recess 76 that extends axially from the distal end 42 of the plunger rod 40 through at least a portion of the body 52 of the plunger rod 40 towards the proximal end 50 of the plunger rod 40. The elongated recess 76 is sized to receive the guide member 60 as the second stopper 28 moves through the barrel 16 in the distal direction towards the first stopper 26. More specifically, once the first stopper 26 is seated in its distal-most position (shown in FIG. 2D), the practitioner can continue to press on the plunger rod 40 causing the second stopper 28 to move in the distal direction. Distal movement of the second stopper 28 causes the second stopper 28 to slide along the elongated guide member 60, meaning that the guide member 60 is received in the elongated recess 76 of the plunger rod 40. In FIG. 2E, the second stopper 28 is in its distal-most position, in which it contacts or nearly contacts the proximal end or proximal surface 32 of the first stopper 26, and the guide member 60 is fully received within the elongated recess 76 of the plunger rod 40. In some examples, the guide member 60 also includes an axially extending slot, locking member, or recess 78 that receives the tab 48 of the plunger rod 40. The engagement between the tab 48 of the plunger rod 40 and the recess 78 of the guide member 60 prevents the plunger rod 40 from rotating about the guide member 60, meaning that any rotation of the plunger rod 40 is transferred to the first stopper 26 through the guide member 60.

As previously described, fluid communication between the second chamber 14 and the first chamber 12 of the syringe 10 is established by rotating the first stopper 26 relative to the syringe barrel 16. Specifically, the practitioner can grasp the plunger rod 40 and rotate the plunger rod 40, which causes the first stopper 26 and the second stopper 28 to rotate relative to the syringe barrel 16. Structures of the first stopper 26 and the syringe barrel 16 that provide the fluid communication between the chambers 12, 14 of the syringe 10 through the first stopper 26 will now be described in further detail.

With reference to FIGS. 3A-3C, the first stopper 26 includes the channels 30 for allowing fluid to pass between the second chamber 14 and the first chamber 12 of the syringe barrel 16. As shown in FIGS. 3A-3C, the first stopper 26 includes two channels positioned on opposite sides of the first stopper 26, such that the first stopper 26 is vertically symmetrical (i.e., symmetrical through a vertical plane). In other examples, the first stopper 26 can include more than two channels 30, such as channels 30 positioned at the 0 degree, 90 degree, 180 degree, and 270 degree positions about the periphery of the first stopper 26. In other examples, the first stopper 26 can include only a single channel 30. The channels 30 include an inflow portion or inlet 80, which receives the second fluid F2 from the second chamber 14, and an outflow portion or outlet 82 for expelling the second fluid F2 from the channels 30 into the first chamber 12. As shown in FIGS. 3A-3C, the channels 30 are cutouts or recesses that extend axially from the proximal end or proximal surface 32 of the first stopper 26 and radially inwardly from the outer peripheral surface 36 of the first stopper 26.

As shown in FIGS. 3B and 3C, the first stopper 26 can also include a channel or groove 84 on the distal surface 34 of the first stopper 26 positioned so that fluid from the second chamber 14 can pass through the first chamber 12 to the fluid port 22 at the distal end 20 of the syringe barrel 16. For example, the channel or groove 84 can be an elongated recess positioned on a center of the distal surface 34 of the first stopper 26 positioned so that the first stopper 26 does not block fluid from passing into the fluid port 22 at the distal end 20 of the barrel 16.

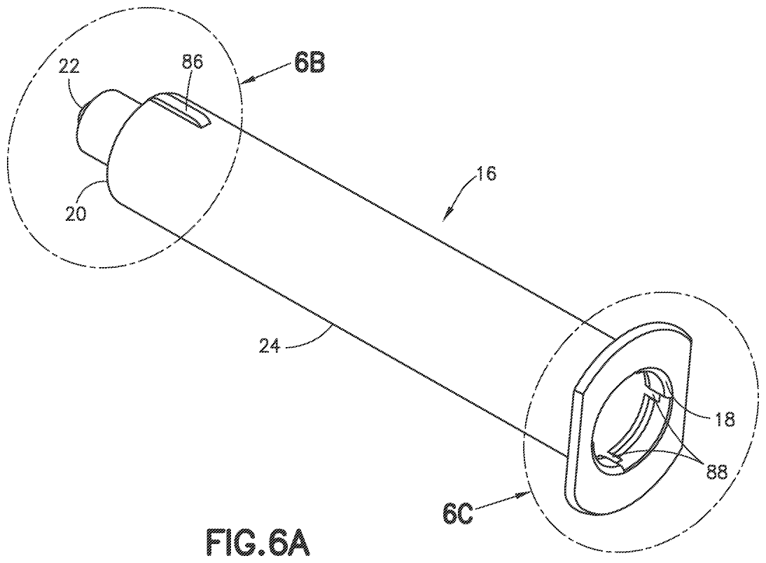

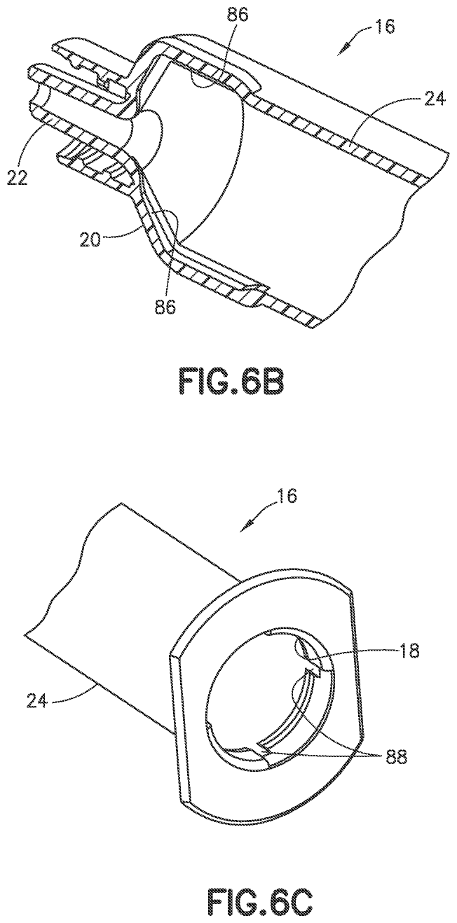

With reference to FIGS. 6A and 6B, the syringe barrel 16 includes one or more radially extending or widened portions 86 proximate to the distal end 20 of the barrel 16 positioned to permit fluid flow through the channels 30 of the first stopper 26 when the first stopper 26 is correctly aligned with the widened portions 86 of the barrel 16. As used herein, the widened spaces or portions 86 of the barrel 16 refer to areas of the barrel 16 where a radial distance between an inner surface of the sidewall 24 of the barrel 16 and a central axis of the barrel 16 is greater than the radial distance between the inner surface of the sidewall 24 and the central axis for other portions of the barrel 16. For example, the widened spaces or portions 86 can be elongated spaces or axial slots that are molded into the sidewall 24 of the barrel 16, as shown in FIGS. 6A and 6B.

The channels 30 of the first stopper 26 and widened portions 86 of the barrel 16 are configured such that fluid passes from the second chamber 14 into the channels 30 through the inlets 80 of the channels 30. For most radial orientations of the first stopper 26, the outlets 82 of the channels 30 are sealed by the inner surface of the sidewall 24 of the barrel 16, thereby preventing fluid from passing from the channels 30 to the first chamber 12. When the first stopper 26 is rotated such that the outlet 82 of the channel 30 is within the widened space or portion 86 of the barrel 16, fluid flow from the second chamber 14 to the first chamber 12 through the channel 30 of the first stopper 26 is permitted. Accordingly, rotation of the first stopper 26 relative to the barrel 16 establishes fluid communication between the second chamber 14 and the first chamber 12 through the channels 30.

As shown in FIG. 6B, the barrel 16 includes two widened spaces or portions 86 that correspond to the two channels 30 of the first stopper 26. The dimensions of the widened spaces or portions 86 generally correspond to a width of the channels 30 of the first stopper 26 and, in particular, can be selected to allow fluid to flow through the syringe barrel 16 between the second chamber 14 and the first chamber 12 at a reasonable rate. If the channels 30 or widened portions 86 are too narrow, fluid may not flow well between the chambers 12, 14 of the barrel 16.

The arcuate width of the widened portions 86 of the barrel 16 also affects how far the first stopper 26 must be rotated in order to establish fluid communication between the second chamber 14 and the first chamber 12. If the arcuate width of the widened portions 86 is too wide, the first stopper 26 may only need to rotate a small amount to establish fluid communication between the chambers 12, 14 of the barrel 16, which could allow for inadvertent actuation of the syringe 10 or fluid leaks between the chambers 12, 14. Accordingly, in some examples, the syringe 10 is configured so that the first stopper 26 should be rotated by about 90 degrees relative to the syringe barrel 16 in order to properly align the channels 30 and widened portions 86 to establish fluid communication between the second chamber 14 and the first chamber 12. In that case, the widened portions 86 of the barrel 16 can have an arcuate width of less than 90 degrees or, for example, between about 45 degrees and 70 degrees. A height of the widened portions 86 is necessarily less than a height of the first stopper 26, so that fluid flow through the widened portions 86 only occurs when the first stopper 26 is rotated to correctly align the channels 30 with the widened portions 86 of the syringe barrel 16.

In some examples, with specific reference to FIGS. 6A and 6C, the syringe barrel 16 can also include a rotation restrictor or rotation restricting structure configured to engage the plunger rod 40 to prevent rotation of the plunger rod 40 relative to the syringe barrel 16 at incorrect times. For example, as shown in FIG. 6C, the barrel 16 can include elongated grooves 88 extending axially along a proximal portion of the sidewall 24 of the barrel 16. The grooves 88 are configured to receive radially extending portions or fins 90 of the plunger rod 40 to prevent the plunger rod 40 from rotating until the first stopper 26 is seated in its distal-most position (shown in FIGS. 2C-2E) within the syringe barrel 16.

More specifically, as shown in FIG. 7A, the plunger rod 40 includes the radially extending fins 90 that extend radially outwardly from a central portion 92 of the plunger rod 40. Near the distal end 42 of the plunger rod 40, the fins 90 extend radially beyond other portions of the plunger rod 40, such that the fins 90 can be received within the axial grooves 88 near the proximal end 18 of the barrel 16. When the fins 90 are received within the grooves 88, the plunger rod 40 is prevented from rotating relative to the syringe barrel 16. Near a middle of the plunger rod 40, the fins 90 include a tapered portion 94 (shown in FIG. 7A) that tapers radially inwardly away from the grooves 88 of the barrel 16, meaning that portions of the fins 90 between the tapered portion 94 at middle the plunger rod 40 and the proximal end 50 of the plunger rod 40 are not received within the grooves 88 of the barrel 16. Accordingly, when the plunger rod 40 is first pushed into the syringe barrel 16, the fins 90 are received within and engage the grooves 88 preventing rotation of the plunger rod 40. When the plunger rod 40 is inserted farther into the barrel 16, such that the tapered portions 94 of the fins 90 pass through the grooves 88, the grooves 88 no longer restrict rotation of the plunger rod 40. Therefore, the plunger rod 40 is free to rotate relative to the barrel 16, meaning that the stoppers 26, 28 can be rotated to establish fluid communication between the second chamber 14 and the first chamber 12.

Method of Expelling Fluid from a Multi-Chamber Syringe

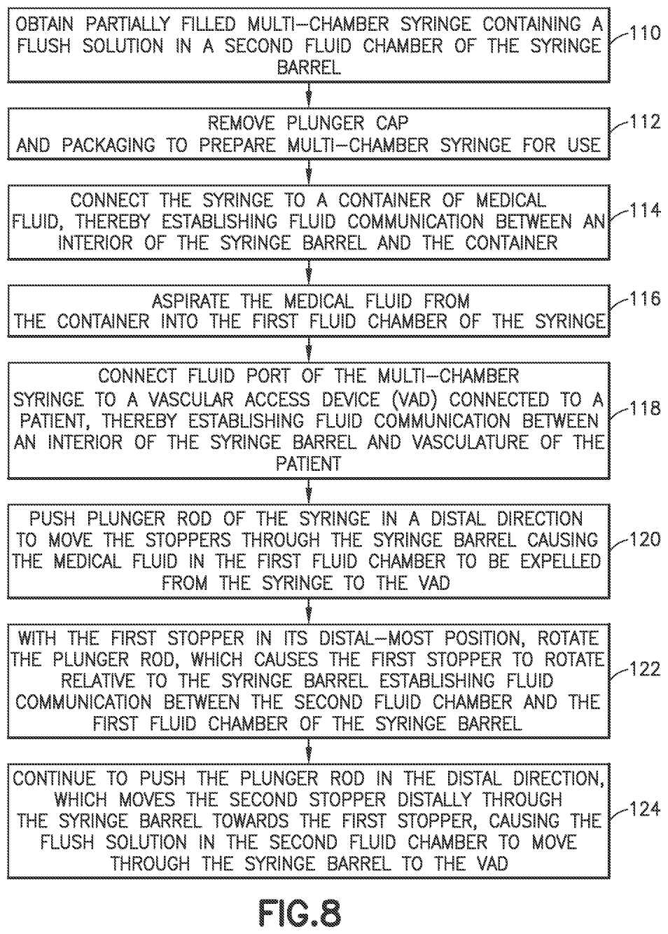

As previously described, the syringe 10 is used for sequential expulsion of fluids, such as expulsion of a medical fluid followed by expulsion of a flush solution from the syringe 10 to the VAD. A flow chart illustrating steps for a sequential fluid expulsion process using the syringe 10 is shown in FIG. 8.

As shown in FIG. 8, at step 110, a practitioner initially obtains a partially filled syringe 10, in which the second chamber 14 of the syringe 10 is filled with a predetermined volume of a second fluid F2, such as the flush solution. For example, the partially filled syringe 10 can contain about 1 mL to about 20 mL or, preferably, about 5 mL to 10 mL of the flush solution. The syringe 10 in the initial or partially-filled position is shown in FIG. 2A. At step 112, the practitioner prepares the syringe 10 for use by, for example, removing any packaging from the syringe 10 and removing the plunger rod cap 58 that holds the plunger rod 40 in place. The practitioner may also move the plunger rod 40 in the distal direction, shown by arrow D in FIGS. 2C and 2D, to fully seat the first stopper 26 of the syringe 10 in its distal-most position. At step 114, the practitioner places the syringe barrel 16 in fluid communication with an interior of a container containing the medical fluid to be delivered to the patient. For example, the practitioner may attach a needle (not shown) to the fluid port 22 at the distal end 20 of the syringe barrel 16 and insert the needle into the container, such as a medical vial, containing the medical fluid to be injected to the patient.

At step 116, the practitioner aspirates the medical fluid into the first chamber 12 of the barrel 16. For example, the practitioner may grasp the plunger rod 40 and move the plunger rod 40 in the proximal direction, shown by arrow P in FIG. 2B, which moves the stoppers 26, 28 in the proximal direction, thereby aspirating or drawing fluid from an interior of the container into the first chamber 12 of the syringe barrel 16, as shown by arrows A2 in FIG. 2B. The syringe 10 is shown in its fully filled position in FIG. 2B with the first fluid F1 in the first chamber 12 and the second fluid F2 in the second chamber 14.

Once a dose of the medical fluid to be delivered to the patient is drawn into the first chamber 12, at step 118, the practitioner removes the needle from the fluid port 22 of the syringe barrel 16 and connects the fluid port 22 to the VAD. For example, the practitioner may insert a nozzle of the syringe barrel 16 into a corresponding port or valve of the VAD, thereby establishing fluid communication between the syringe barrel 16 and a lumen of the VAD. At step 120, once the syringe 10 is appropriately connected to the VAD, the practitioner grasps the plunger rod 40 and pushes the plunger rod 40 in the distal direction, which causes the stoppers 26, 28 to move distally (shown by arrow D in FIGS. 2C and 2D) through the syringe barrel 16. Distal movement of the first stopper 26 causes the first fluid F1 (e.g., the medical fluid) in the first chamber 12 of the syringe barrel 16 to be expelled from the syringe barrel 16, as shown by arrow A3 in FIG. 2C, to the VAD through the fluid port 22 or nozzle of the syringe barrel 16. The practitioner continues to move the plunger rod 40 in the distal direction until the first stopper 26 is seated in the syringe barrel 16 at its distal-most position. The first stopper 26 is shown in this distal-most position in FIG. 2C.

At step 122, with the first stopper 26 in its distal-most position, the practitioner rotates the first stopper 26 (as shown by arrow A1 in FIG. 2D) by a predetermined amount, such as by about 90 degrees, in order to properly align the outlets 82 of the channels 30 of the first stopper 26 with the widened portions 86 or spaces of the syringe barrel 16. As previously described, rotating the first stopper 26 relative to the syringe barrel 16 in this manner establishes fluid communication between the second chamber 14 and the first chamber 12 of the syringe barrel 16, as shown by arrow A4 in FIG. 2D.

At step 124, once fluid communication between the second chamber 14 and the first chamber 12 is established, the practitioner continues to push the plunger rod 40 in the distal direction, which moves the second stopper 28 through the syringe barrel 16 in the distal direction towards the proximal surface 32 of the first stopper 26. Movement of the second stopper 28 towards the first stopper 26 causes the second fluid F2, such as the flush solution, in the second chamber 14 to pass into the channels 30 of the first stopper 26 and into the first chamber 12. The second fluid F2 then passes through the first chamber 12 and is expelled from the syringe barrel 16 to the VAD through the fluid port 22 at the distal end 20 of the syringe barrel 16. The syringe 10 is shown in an end-of-use or final position in FIG. 2E, with the distal surface 34 of the first stopper 26 in contact with or nearly contacting the proximal surface 32 of the first stopper 26.

Multi-Chamber Syringe Including a Stopper with an Enclosed Fluid Passageway

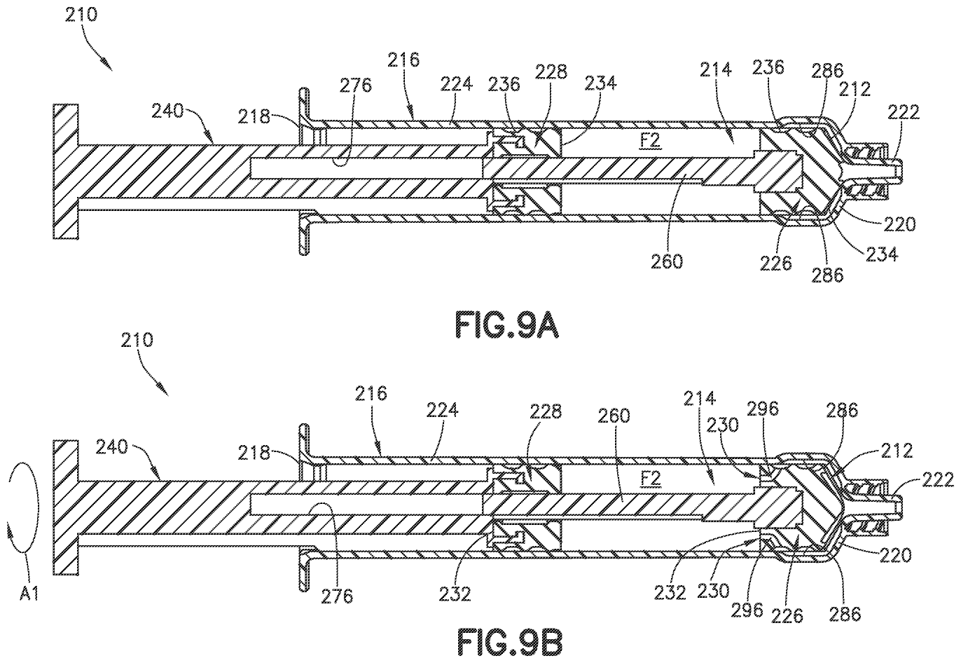

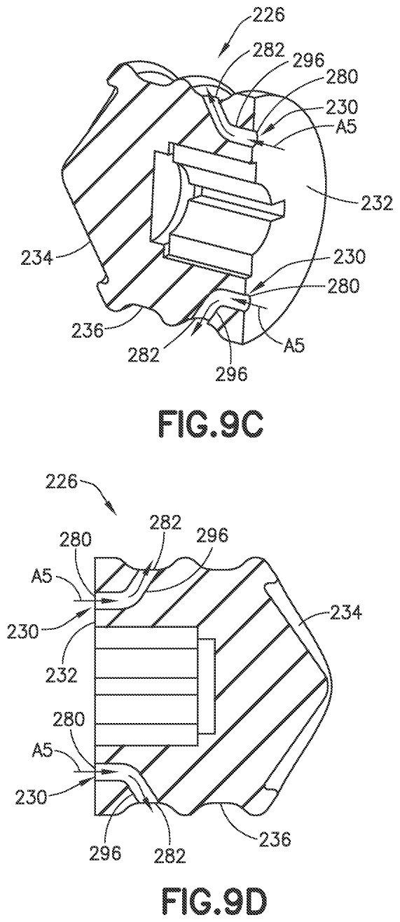

FIGS. 9A-9D illustrate another example of a multi-chamber syringe 210 for sequential delivery of a first fluid F1, such as a medical fluid, followed by a second fluid F2, such as a flush solution. The syringe 210 includes many of the components and features of the syringe 10 shown in FIGS. 1A-2E. In particular, the syringe 210 comprises the barrel 216 having an open proximal end 218, a distal end 220 including a fluid port 222, and a sidewall 224 extending between the proximal end 218 and the distal end 220 of the barrel 216. The syringe 210 further comprises the first stopper 226 and the second stopper 228 slideably positioned in the barrel 216. The stoppers 226, 228 separate the barrel 216 into chambers 212, 214. Specifically, the syringe 210 includes a first chamber 212 between the distal end 220 of the barrel 216 and a distal surface 234 of the first stopper 226 and a second chamber 214 between a proximal surface 232 of the first stopper 226 and the second stopper 228.

As in previous examples, the syringe 210 also includes the plunger rod 240 connected to the second stopper 228 for moving the second stopper 228 and/or the first stopper 226 through the barrel 216. The syringe 210 also includes the guide member 260 fixed to the first stopper 226 for rotating the first stopper 226 relative to the barrel 216. As in previous examples, rotation of the plunger rod 240 causes the guide member 260 to rotate which, in turn, causes rotation of the first stopper 226. Also as in previous examples, the plunger rod 240 includes the elongated recess 276, which receives the guide member 260 when the second stopper 228 is moved in the distal direction towards the first stopper 226.

As in previous examples, the first stopper 226 includes channels 230 for establishing fluid communication between the second chamber 214 and the first chamber 212. Specifically, rotation of the first stopper 226 relative to the syringe barrel 216 establishes the fluid communication between the second chamber 214 and the first chamber 212 through the channel 230 of the first stopper 226. The syringe 210 differs from previous examples in the structure of the channels 230. Specifically, in contrast to the cutout portions or recess of previous examples, the channels 230 of the first stopper 226 are enclosed passages 296 extending from an inlet 280 on the proximal surface 232 of the first stopper 226 to an outlet 282 on the outer surface 236 of the first stopper 226. As shown in FIGS. 9A-9D, the first stopper 226 includes two enclosed passages 296 on opposite sides of the first stopper 226, such that the first stopper 226 is vertically symmetrical. In other examples, the first stopper 226 can include only a single enclosed passage 296 or more than two enclosed passages 296. For example, the first stopper 226 could include enclosed passages 296 at the 0 degree, 90 degree, 180 degree, and 270 degree positions about the periphery of the first stopper 226 or at any other convenient position on the first stopper 226.

The syringe 210 operates in the same manner as previous examples. Specifically, once the syringe 210 is fully filled with the first fluid F1 in the first chamber 212 and the second fluid F2 in the second chamber 214, the fluids F1, F2 are expelled from the syringe 210 by moving the plunger rod 240 in the distal direction. Distal movement of the plunger rod 240 causes the first stopper 226 and the second stopper 228 to move distally through the syringe barrel 216. As in previous examples, the first fluid F1 is expelled from the first chamber 212 through the fluid port 222 of the barrel 216 until the first stopper 226 is seated in its distal-most position against the distal end 220 of the barrel 216, as shown in FIG. 9A. Next, as in previous examples, the practitioner rotates the plunger rod 240, as shown by arrow A1 in FIG. 9B, which causes the outlets 282 of the channels 230 to move into the widened portions 286 of the barrel 216, thereby establishing fluid communication between the second chamber 214 and the first chamber 212.

Once the outlets 282 are in the widened portions 286 of the barrel 216, the second fluid F2 is able to move through the channels 230, as shown by arrows A5 in FIGS. 9C and 9D, to the first chamber 212. Continued distal movement of the plunger rod 240 causes the second stopper 228 to move along the guide member 260 toward the first stopper 226, which causes the second fluid F2 to pass from the second chamber 214, through the channels 230, into the first chamber 212, and then from the barrel 216 through the fluid port 222. Accordingly, the syringe 210 provides for sequential expulsion of the first fluid F1 followed by the second fluid F2 with a single continuous movement of the plunger rod 240 in the distal direction.

While examples of the multi-chamber syringes and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A multi-chamber syringe for sequential expulsion of at least a first fluid followed by a second fluid, the syringe comprising:
a barrel comprising a proximal end, a distal end comprising a fluid port for expulsion of the first fluid and the second fluid from the barrel, and a sidewall extending between the proximal end and the distal end of the barrel;
a first stopper comprising at least one channel, the first stopper being slidably positioned in the barrel with a first chamber configured to contain the first fluid between the distal end of the barrel and the first stopper; and
a second stopper slidably positioned in the barrel proximal to the first stopper with a second chamber configured to contain the second fluid between the first stopper and the second stopper,
wherein rotation of the first stopper relative to the syringe barrel establishes fluid communication between the second chamber and the first chamber through the at least one channel of the first stopper.

2. The syringe of claim 1, wherein movement of the first stopper and the second stopper through the barrel in a proximal direction aspirates fluid into the first chamber through the fluid port of the barrel.

3. The syringe of claim 1, wherein, in a filled position with the first fluid in the first chamber and the second fluid in the second chamber, the first stopper is spaced apart from the second stopper and the second chamber is not in fluid communication with the first chamber, and
wherein advancing the first stopper and the second stopper through the barrel in a distal direction from the filled position to an intermediate position expels the first fluid from the first chamber through the fluid port of the barrel.

4. The syringe of claim 3, wherein, in the intermediate position, the first stopper is in a distal-most position within the syringe barrel and all or substantially all of the first fluid is expelled from the first chamber, and
wherein, with the stoppers in the intermediate position, rotating the first stopper establishes fluid communication between the second chamber and the first chamber through the at least one channel of the first stopper.

5. The syringe of claim 4, wherein, with the second chamber in fluid communication with the first chamber, movement of the second stopper axially through the barrel towards the first stopper moves the second fluid from the second chamber, through the at least one channel of the first stopper, the first chamber, and the fluid port of the barrel, thereby expelling the second fluid from the barrel.

6. The syringe of claim 1, wherein the barrel comprises at least one widened portion, wherein a radial distance between an inner surface of the sidewall of the barrel in the at least one widened portion and a central axis of the barrel is greater than the radial distance between the inner surface of the sidewall and the central axis of the barrel at other portions of the barrel.

7. The syringe of claim 6, wherein the at least one channel through the first stopper comprises an inlet and an outlet, and wherein fluid flow through the at least one channel occurs only when the outlet of the at least one channel is in the at least one widened portion of the barrel.

8. The syringe of claim 1, wherein the at least one channel of the first stopper comprises a first cutout section on one side of the stopper and a second cutout section on an opposite side of the first stopper from the first cutout section, such that the first stopper is vertically symmetrical.

9. The syringe of claim 1, wherein the at least one channel comprises at least one enclosed passage extending from an inlet on a proximal end of the first stopper to an outlet on an outer peripheral surface of the first stopper.

10. The syringe of claim 1, wherein the first stopper comprises a recessed groove on the distal end of the first stopper positioned to permit fluid flow past the first stopper to the fluid port of the barrel.

11. The syringe of claim 1, further comprising at least one guide member fixed to the first stopper for rotating the first stopper relative to the barrel,
wherein a distal end portion of the at least one guide member is received within a cavity of the first stopper, and
wherein the at least one guide member comprises at least one axially extending ridge that engages an axial slot of the cavity, thereby fixing the at least one guide member to the first stopper.

12. The syringe of claim 11, wherein the second stopper comprises a through-hole, and wherein the at least one guide member extends through the through-hole, allowing the second stopper to slide along the at least one guide member when the second stopper is moved through the barrel in a distal direction.

13. The syringe of claim 11, further comprising a plunger rod connected to the second stopper for moving the second stopper and/or the first stopper through the barrel.

14. The syringe of claim 13, further comprising a removable plunger cap engaged between the plunger rod and the barrel for preventing movement of the plunger rod until the removable plunger cap is removed from the syringe.

15. The syringe of claim 13, wherein the plunger rod comprises an elongated recess that receives the at least one guide member as the second stopper moves through the barrel in a distal direction towards the first stopper.

16. The syringe of claim 13, wherein the barrel comprises at least one rotation restrictor that engages the plunger rod preventing rotation of the plunger rod, stoppers, and the at least one guide member unless the first stopper is at a distal-most position in the barrel.

17. A pre-filled flushing syringe, comprising:
a barrel comprising a proximal end, a distal end comprising a fluid port for expulsion of the first fluid and the second fluid from the barrel, and a sidewall extending between the proximal end and the distal end of the barrel;
a first stopper comprising at least one channel, the first stopper being slidably positioned in the barrel with a first chamber configured to contain the first fluid between the distal end of the barrel and the first stopper; and
a second stopper slidably positioned in the barrel proximal to the first stopper with a second chamber configured to contain the second fluid between the first stopper and the second stopper,
wherein rotation of the first stopper relative to the syringe barrel establishes fluid communication between the second chamber and the first chamber through the at least one channel of the first stopper; and
a predetermined volume of the second fluid disposed in the second chamber of the pre-filled syringe,
wherein the pre-filled syringe is provided with the first chamber fluidly isolated from the second chamber, thereby containing the predetermined volume of the second fluid within the second chamber of the pre-filled syringe.

18. The pre-filled syringe of claim 17, wherein the second fluid comprises a saline and/or heparin flush solution.

19. A method for sequential expulsion of fluids from the syringe of claim 1, the method comprising:
moving the first stopper and the second stopper of the syringe of claim 1 through the barrel in a distal direction, thereby expelling the first fluid from the barrel through the fluid port of the barrel;
rotating the first stopper, thereby establishing fluid communication between the second chamber and the first chamber; and
moving the second stopper through the barrel in the distal direction towards the first stopper, thereby causing the second fluid to pass from the second chamber, through the at least one channel of the first stopper, the first chamber, and the fluid port of the barrel.

20. The method of claim 19, wherein the syringe is provided partially prefilled containing a predetermined volume of the second fluid in the second chamber.
